(19) United States
(12) Patent Application Publication
(10) Pub. No.: US 2025/0093261 A1
(43) Pub. Date: Mar. 20, 2025

(54) LINE-ILLUMINATING LIGHT SOURCE FOR IMAGING APPARATUS, IMAGING APPARATUS INCLUDING THE SAME, AND SKIN ANALYZING APPARATUS INCLUDING THE IMAGING APPARATUS

(71) Applicants: SAMSUNG ELECTRONICS CO., LTD., Suwon-si (KR); CHUNG ANG University Industry Academic Cooperation Foundation, Seoul (KR)

(72) Inventors: Unjeong KIM, Suwon-si (KR); Hojung KIM, Suwon-si (KR); Hyungbin SON, Seoul (KR); Suyeon LEE, Suwon-si (KR)

(73) Assignees: SAMSUNG ELECTRONICS CO., LTD., Suwon-si (KR); CHUNG ANG University Industry Academic Cooperation Foundation, Seoul (KR)

(21) Appl. No.: 18/887,698

(22) Filed: Sep. 17, 2024

(30) Foreign Application Priority Data
Sep. 18, 2023 (KR) 10-2023-0124307

(51) Int. Cl. G01N 21/33; G01N 21/17; G01N 21/27; G01N 21/47

(52) U.S. Cl. CPC G01N 21/33; G01N 21/27; G01N 21/4738; G01N 2021/1765; G01N 2021/4764; G01N 2201/0636; G01N 2201/0638; G01N 2201/064; G01N 2201/0686

(57) ABSTRACT

An imaging apparatus may include a light source emitting linear light, a light reflector configured to reflect the linear light incident from the light source unit, a first filter configured to filter the linear light reflected from the light reflector, a light transmission reflector configured to reflect the linear light passing through the first filter toward an object and transmit light incident from the object, a second filter configured to filter the light that has passed through the light transmission reflector after being incident from the object, and a camera configured to capture the light passing through the second filter.

### CROSS-REFERENCE TO RELATED APPLICATION

This application claims priority to Korean Patent Application No. 10-2023-0124307, filed on Sep. 18, 2023, in the Korean Intellectual Property Office, the disclosure of which is incorporated by reference herein in its entirety.

### BACKGROUND

### 1. Field

The disclosure relates to an imaging apparatus, and more particularly, to a line-illuminating light source for an imaging apparatus for obtaining images for skin condition analysis, an imaging apparatus including the same, and a skin analyzing apparatus including the imaging apparatus.

### 2. Description of the Related Art

As interest in skin beauty and skin care increases, various skin analyzing apparatuses have been introduced to analyze skin conditions. With regard to skin analyzing apparatuses that have been developed, skin analysis is typically performed from skin images obtained by radiating light (e.g., ultraviolet rays) to a face or skin.

### SUMMARY

One or more embodiments may provide an imaging apparatus that may minimize exposure of an object to ultraviolet light.

Further, one or more embodiments may provide an imaging apparatus that may prevent the decrease in the intensity of light received by a camera from an object.

The imaging apparatus may include a line-illuminating light source configured to emit light along a linear path, instead of a surface light source configured to emit light uniformly across a surface, to minimize the amount of light emitted onto the object. The line-illuminating light source may be also referred to as a line light source or a linear light source. The surface light source may be also referred to as an area light source or a planar light source.

Still further, one or more embodiments may provide a skin analyzing apparatus including the imaging apparatus.

Additional aspects will be set forth in part in the description which follows and, in part, will be apparent from the description, or may be learned by practice of the presented embodiments of the disclosure.

According to an aspect of the present disclosure, a line-illuminating light source may include: a light emitter; a first lens provided at a position where light emitted from the light emitter is incident; a slit including a linear opening through which the light incident from the first lens passes; and a second lens provided at a position where the light passing through the linear opening is incident, and configured to direct the light toward an object.

The first lens may be an aspherical lens.

The second lens may be an achromatic lens.

The light emitter may be configured to emit ultraviolet light.

The light emitter, the first lens, the slit, and the second lens may be aligned on a same optical axis.

According to another aspect of the present disclosure, an imaging apparatus may include: a light source configured to emit linear light; a light reflector configured to reflect the linear light incident from the light source; a first filter configured to filter the linear light reflected from the light reflector; a light transmission reflector configured to reflect the linear light passing through the first filter toward an object and transmit light incident from the object; a second filter configured to filter the light that has passed through the light transmission reflector after being incident from the object; and a camera configured to capture the light passing through the second filter, wherein the light transmission reflector, the second filter, and the camera may be aligned on a same optical axis.

The light source may include: a light emitter; a first lens provided at a position where the light emitted from the light emitter is incident; a slit including a linear opening through which the light incident from the first lens passes; and a second lens provided at a position where the light passing through the linear opening is incident.

The light emitter may be configured to emit ultraviolet light.

The camera may include a hyperspectral image sensor.

The light reflector may be configured to rotate about two axes perpendicular to each other so that the linear light incident from the light source moves toward the object in two different perpendicular directions.

The light transmission reflector may be configured to reflect ultraviolet rays and transmit visible light.

The light source, the light reflector, the first filter, and the light transmission reflector may be aligned on a same optical axis.

The light source, the light reflector, the first filter, the light transmission reflector, the second filter, and the camera may be aligned so that light reflected from the light transmission reflector and radiated to the object and light incident from the object to the light transmission reflector due to the radiated light are parallel to each other.

The light reflector may include a folding mirror.

The light reflector may include a dichroic mirror.

The first filter may include an excitation filter.

The second filter may include an emission filter.

A skin analyzing apparatus may include: a mask structure configured to cover a surface of an object; and the imaging apparatus provided in an region of the mask structure facing the surface of the object.

In one example, the imaging apparatus may be mounted in the mask structure.

In one example, the imaging apparatus may be attachable to and detachable from the mask structure.

### BRIEF DESCRIPTION OF THE DRAWINGS

The above and other aspects will be more apparent by describing certain example embodiments, with reference to the accompanying drawings, in which:

FIG. 1 is a block diagram showing a hyperspectral imaging system according to an embodiment;

FIG. 2 is a perspective view illustrating linear light emitted from a light source unit;

FIG. 3 is a view illustrating linear light that moves up and down on an object when a light reflection unit of FIG. 1 is rotated about a first axis;

FIG. 4 is a view illustrating linear light that moves left and right on an object when the light reflection unit of FIG. 1 is rotated about a second axis;

FIG. 5 illustrates a case in which a selected area of an object in FIG. 1 is line-scanned by linear light emitted from a light-emitting unit;

FIG. 6 is a block diagram showing an example of the light source unit of FIG. 1;

FIG. 7 illustrates an example of a slit of FIG. 6; and

FIG. 8 is a cross-sectional view showing a skin condition analysis apparatus according to an embodiment.

### DETAILED DESCRIPTION

Example embodiments are described in greater detail below with reference to the accompanying drawings.

In the following description, like drawing reference numerals are used for like elements, even in different drawings. The matters defined in the description, such as detailed construction and elements, are provided to assist in a comprehensive understanding of the example embodiments. However, it is apparent that the example embodiments can be practiced without those specifically defined matters. Also, well-known functions or constructions are not described in detail since they would obscure the description with unnecessary detail.

As used herein, the term “and/or” includes any and all combinations of one or more of the associated listed items. Expressions such as “at least one of,” when preceding a list of elements, modify the entire list of elements and do not modify the individual elements of the list. While such terms as “first,” “second,” etc., may be used to describe various elements, such elements must not be limited to the above terms. The above terms may be used only to distinguish one element from another.

Hereinafter, a line-illuminating light source for an imaging apparatus, the imaging apparatus including the same, and a skin analyzing apparatus including the imaging apparatus will be described more fully with reference to the accompanying drawings. In the drawings, thicknesses of layers and regions may be exaggerated for clarification of the specification.

The embodiments of the disclosure are capable of various modifications and may be embodied in many different forms. In a layer structure described below, when a position of an element is described using an expression “above” or “on”, the position of the element may include not only the element being “immediately in a contact manner” but also being in a non-contact manner”. In the following descriptions, like reference numerals in the drawings refer to like elements throughout,

The singular forms include the plural forms unless the context clearly indicates otherwise. When a portion “includes” an element, another element may be further included, rather than excluding the existence of the other element, unless otherwise described.

The term “above” and similar directional terms may be applied to both singular and plural. With respect to operations that constitute a method, the operations may be performed in any appropriate sequence unless the sequence of operations is clearly described or unless the context clearly indicates otherwise. The operations may not necessarily be performed in the order of sequence.

Also, in the specification, the terms “units” or “ . . . modules” denote units or modules that process at least one function or operation, and may be realized by hardware, software, or a combination of hardware and software.

Connections or connection members of lines between components shown in the drawings illustrate functional connections and/or physical or circuit connections, and the connections or connection members may be represented by replaceable or additional various functional connections, physical connections, or circuit connections in an actual apparatus.

All examples or example terms are simply used to explain in detail the technical scope of the disclosure, and thus, the scope of the disclosure is not limited by the examples or the example terms as long as it is not defined by the claims.

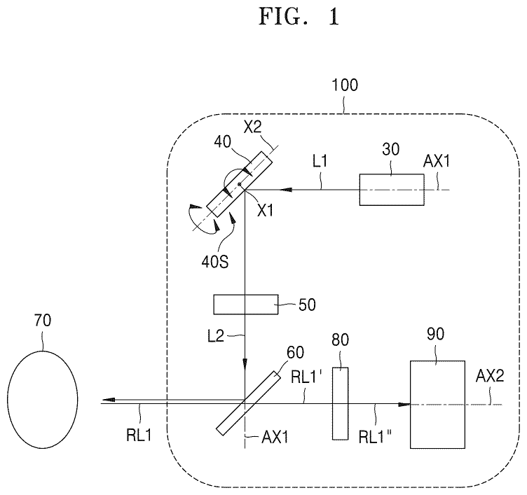

FIG. 1 is a block diagram showing a hyperspectral imaging system 100 according to an embodiment.

Referring to FIG. 1, the hyperspectral imaging system 100 according to an embodiment may include a light source unit (e.g., a light source) 30, a light reflection unit (e.g., a light reflector) 40, a first filter 50, a light transmission reflection unit (e.g., a light transmission reflector) 60, a second filter 80, and a camera 90, but is not limited thereto. The light source unit 30, the light reflection unit 40, the first filter 50, and the light transmission reflection unit 60 may be disposed on the same first optical axis AX1. The light transmission reflection unit 60, the second filter 80, and the camera 90 may be disposed on the same second optical axis AX2. On the first optical axis AX1, the light reflection unit 40 and the first filter 50 may be provided between the light source unit 30 and the light transmission reflection unit 60. The light reflection unit 40 may be disposed between the light source unit 30 and the first filter 50. The first filter 50 may be disposed between the light reflection unit 40 and the light transmission reflection unit 60. On the second optical axis AX2, the second filter 80 may be provided between the light transmission reflection unit 60 and the camera 90. In other words, the light transmission reflection unit 60, the second filter 80, and the camera 90 may be aligned so that light RL1 reflected from an object 70 is incident on the camera 90 through the light transmission reflection unit 60 and the second filter 80. The light source unit 30, the light reflection unit 40, the first filter 50, and the light transmission reflection unit 60 may be aligned so that light L1 emitted from the light source unit 30 is incident on the object 70 through the light reflection unit 40, the first filter 50, and the light transmission reflection unit 60. The light transmission reflection unit 60 may be located at a point where the first optical axis AX1 and the second optical axis AX2 intersect.

In one example, the light source unit 30 may include a light source (hereinafter referred to as a line-illuminating light source, line source, or line light source) that emits linear light or line light, but is not limited thereto. In one example, the light source unit 30 may include a line-illuminating light source that emits ultraviolet rays (UV), but the wavelength of the emitted light is not limited to the UV range. For example, the light source unit 30 may include a light source that emits ultraviolet rays at a specific wavelength or within a specific wavelength band of ultraviolet rays. In one example, the ultraviolet rays may generate fluorescence (visible light) when radiated to the object 70. For example, the ultraviolet rays may include ultraviolet rays of 365 nm wavelength, but are not limited thereto. In one example, the light source unit 30 may include a mercury lamp, a laser light source, or a light-emitting diode (LED) that emits ultraviolet rays, but is not limited to these light sources. The LED may include a high output LED.

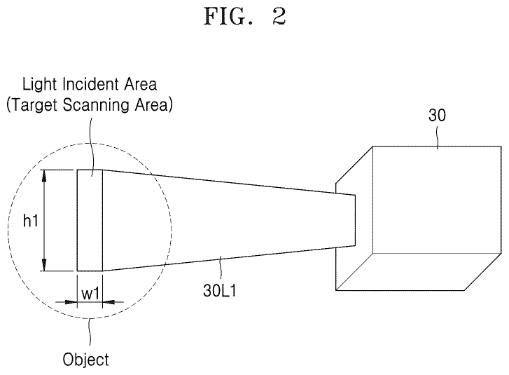

FIG. 2 shows linear light 30L1 emitted from the light source unit 30 and incident onto an object. In FIG. 2, when the linear light 30L1 is cut in a direction perpendicular to the direction in which the linear light 30L1 travels, a cross-section of a light incident area of the linear light 30L1 may have a shape of a rectangle with different lengths in perpendicular directions. If a relatively short length is called a width w1 and a relatively long length is called a height h1, the cross section may be a rectangle having the height h1 much greater than the width w1, and therefore, the cross section may be expressed as a linear line or line. Therefore, the light L1 emitted from the light source unit 30 of FIG. 1 may be expressed as linear light, line-shaped light, or line light. In one example, the width w1 may be in a range from about 1 mm to about 3 mm, but is not limited thereto. In one example, the height h1 may be in a range from about 50 mm to about 100 mm, but is not limited thereto.

In one example, the width w1 and the height h1 may differ from the above range depending on the size of a region of interest (area to be analyzed) of the object 70. For example, when the height h1 of the region of interest of the object 70 is between 10 mm and 50 mm, the height h1 of the linear light 30L1 may have a height corresponding to this range.

In one example, the light reflection unit 40 provided to reflect the light L1 emitted from the light source unit 30 to the first filter 50 may be provided to rotate about two axes X1 and X2. At this time, a range of rotation about each axis X1 and X2 may be limited. For example, the range of rotation may be limited to an acute angle. For example, the light reflection unit 40 may be provided to rotate within an acute angle about the first axis X1 and may be rotated within an acute angle about the second axis X2 perpendicular to the first axis X1. As the light reflection unit 40 rotates around the two axes X1 and X2, the hyperspectral imaging system 100 may perform two-dimensional scanning of the object 70, and because the light emitted from the light source unit 30 is linear light, the object 70 may be scanned by radiating linear light only to a selected target scanning area to be photographed (selected region to be imaged) without radiating the linear light to the entire object 70 or a wide region of the object 70. The object 70 may be a human face or skin, or may be the skin of another living things. In one example, if the object 70 is a person's face or skin, the light radiated to the object 70 may be or include ultraviolet rays (UV) for checking or analyzing the condition of the person's face or skin. In this case, in order to prevent or minimize damage (wounds) to the object 70 due to ultraviolet radiation, the region of the object 70 exposed to ultraviolet rays may be limited to a region of interest of the object 70. The illustrated hyperspectral imaging system 100 may radiate ultraviolet rays only to the target scanning area of the object 70 based on the operation of the light reflection unit 40 as described above, thereby preventing or minimizing damage to the object 70 due to ultraviolet radiation. In one example, the light reflection unit 40 may be or include a folding mirror, but is not limited thereto. In one example, a reflective surface 40S of the light reflection unit 40 may be parallel to a plane consisting of the two axes X1 and X2.

The first filter 50 may include a filter equipped to pass light of a specific band (or specific wavelength) of the light L1 that is emitted from the light source unit 30, reflected by the light reflection unit 40, and incident on the first filter 50 and to block the rest of the light L1. In one example, the first filter 50 may be or include an excitation filter that passes ultraviolet rays of a first wavelength and blocks the rest of the light L1. In one example, the first wavelength may be 365 nm, but is not limited thereto. The light transmission reflection unit 60 may be an optical element or

may include the optical element having an optical characteristic of reflecting part of the incident light and transmitting the rest. At this time, the incident light may include light of different wavelengths. Accordingly, the light transmission reflection unit 60 may reflect light L2 that is incident passing through the first filter 50 to the object 70. As the light L2 is radiated to the object 70, the light RL1 may be generated (emitted) from the object 70. In one example, the light RL1 may include the light L2 reflected from the object 70, and may include fluorescence of a visible light component emitted as a result of the light L2 being radiated to the object 70. Accordingly, the component of the light RL1 emitted from the object 70 may vary depending on a surface condition of the object 70 or a surface region of the object 70. That is, the light RL1 emitted from the object 70 may include several components of visible light. Therefore, the state of a surface of the object 70 may be analyzed through analysis of the light RL1 emitted from the object 70. For example, when the object 70 is a person's face or skin, the condition of the face or skin may be analyzed by analyzing the light RL1 emitted from the object 70. The light RL1 may include an image of a target scanning area of the object 70, wherein the target scanning area may cover the entire region or a partial region of the object 70. When the light RL1 is sensed with an RGB camera, the image may be an RGB image, and when the light RL1 is sensed with a hyperspectral camera, the image may be a hyperspectral image.

The light RL1 generated from the object 70 may be incident on the camera 90 sequentially through the light transmission reflection unit 60 and the second filter 80. In one example, the light transmission reflection unit 60 may be disposed at an angle with respect to a horizontal plane, for example, at an angle of about 45° with respect to the horizontal plane, but is not limited thereto. In one example, the light transmission reflection unit 60 may be or include a dichroic mirror having the optical properties described above, but is not limited to such a mirror. If the light transmission reflection unit 60 includes a dichroic mirror, the dichroic mirror may include a reflection-transmission layer configured to reflect ultraviolet rays and transmit visible light. Accordingly, the light RL1 emitted from the object 70 may pass through the light transmission reflection unit 60 and be incident on the second filter 80.

In one example, the second filter 80 may include a filter that blocks ultraviolet ray components and passes only visible light among the light incident thereon. Therefore, if light RL1′ that has passed through the light transmission reflection unit 60 includes ultraviolet ray components, the ultraviolet ray components may be removed by the second filter 80. In one example, the second filter 80 may be or include an emission filter.

Light RL1″ that passes through the second filter 80 may include at least one visible light component. As an example, the light RL1″ may include visible light of red (R), green (G), and blue (B) components. In addition, the light RL″ may further include component(s) between red and green, component(s) between green and blue, component(s) between infrared and red, and/or component(s) between blue and ultraviolet. As an example, the light RL1″ may include various components belonging to the visible light band.

The light RL1″ that has passed through the second filter 80 is incident on the camera 90. In one example, the camera 90 may be or include a hyperspectral camera that includes a hyperspectral image sensor. Therefore, a hyperspectral image of a surface of the object 70 may be obtained using the camera 90.

Because the light transmission reflection unit 60, the second filter 80, and the camera 90 are aligned on the same optical axis AX2, a direction in which the light L1 reflected by the light transmission reflection unit 60 is incident on the object 70 and a direction of the camera 90 facing the object 70 may coincide with each other. Accordingly, the reduction of the intensity of light incident on the camera 90 due to the mismatch of the directions may be prevented.

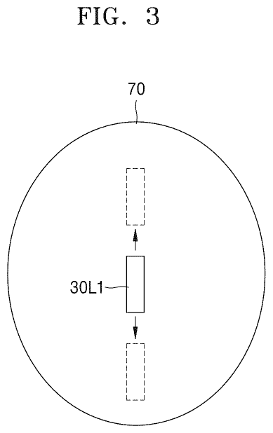

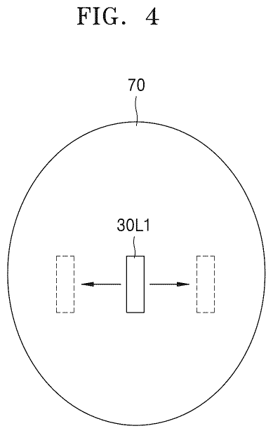

FIGS. 3 and 4 show a case in which linear light 30L1 radiated from the light source unit 30 to the object 70 moves according to the rotation of the light reflection unit 40.

FIG. 3 shows a case that, when the light reflection unit 40 in FIG. 1 is rotated about the first axis X1, the light incident area of the linear light 30L1 (i.e., the light L2 that travels toward the object 70 after being reflected by the light transmission reflection unit 60) moves up and down on the object 70.

FIG. 4 shows a case that, when the light reflection unit 40 in FIG. 1 is rotated about the second axis X2, the light incident area of the linear light 30L1 moves left and right on the object 70.

FIGS. 3 and 4 suggest that any target scanning area of the object 70 may be scanned with the linear light 30L1 by the rotation of the light reflection unit 40.

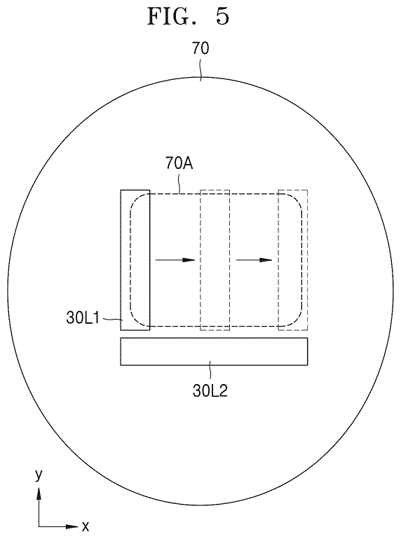

FIG. 5 shows a case in which a selected region 70A of the object 70 is line scanned by linear light 30L1 emitted from the light source unit 30. The selected region 70A may be also referred to as a target scanning area.

Referring to FIG. 5, the selected region 70A may be scanned from left to right by the linear light 30L1. The scanning may also proceed from right to left. Additionally, if the linear light 30L1 is not a vertical linear light (having a length in a y-axis direction) but the linear light 30L1 is a horizontal linear light 30L2 (having a length in an x-axis direction), the scanning of the selected region 70A may proceed from top to bottom or bottom to top. By using these linear lights 30L1 and 30L2, ultraviolet rays are radiated only to a region 70A of interest in the object 70, and ultraviolet rays are not radiated to other regions of the object 70, therefore, an area of the object 70 that is unnecessarily exposed to ultraviolet rays may be minimized.

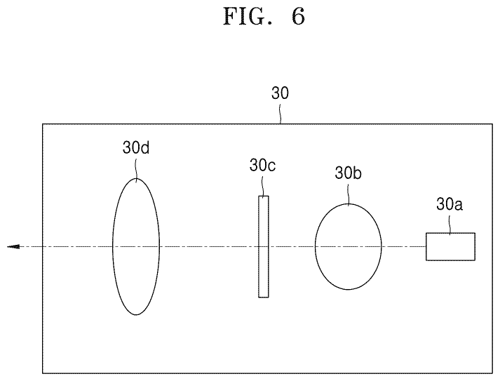

FIG. 6 shows an example of the light source unit 30 of FIG. 1.

Referring to FIG. 6, the light source unit 30 includes a light-emitting unit (e.g., a light emitter) 30a, and a first lens unit 30b, a slit 30c, and a second lens unit 30d sequentially aligned in a direction from the light-emitting unit 30a to the light reflection unit 40, and may further include other elements besides above. In one example, the light-emitting unit 30a may be provided to emit ultraviolet rays and may include a light source described with reference to FIG. 1. In one example, the first lens unit 30b may include an aspherical lens. As an aspherical lens, the first lens unit 30b may include a lens having a light incident surface that is flat and a light exit surface that is aspherical, but is not limited thereto.

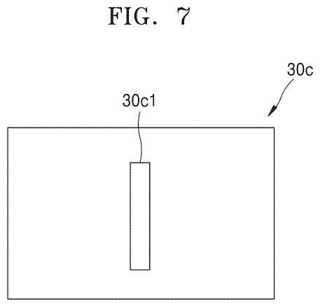

In one example, as shown in FIG. 7, the slit 30c may include a linear opening 30c1 formed to correspond to the linear light 30L1. The light emitted from the light-emitting unit 30a is incident on the slit 30c through the first lens unit 30b, and the light incident on the slit 30c is transmitted to the second lens unit 30d through the linear opening 30c1. The light incident on the second lens unit 30d through the linear opening 30c1 of the slit 30c may be projected onto the light reflection unit 40 as linear light 30L1 while passing through the second lens unit 30d.

In one example, the second lens unit 30d may be or include an achromatic lens. In one example, the second lens unit 30d may include a lens in which a light incident surface is flat and a light exit surface is curved, but is not limited thereto.

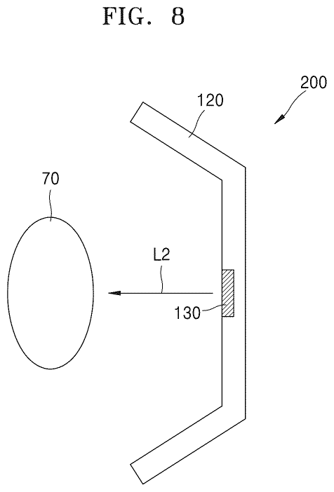

FIG. 8 shows a skin condition analyzing apparatus 200 according to an embodiment.

Referring to FIG. 8, the skin condition analyzing apparatus 200 may include a mask structure 120 configured to cover an entire opposing surface of the object 70 and an imaging apparatus 130 provided on the mask structure 120. The imaging apparatus 130 may be provided in a region of the mask structure 120 facing the object 70 and may be provided to capture (acquire) an image of a surface of the object 70. In one example, the mask structure 120 may have a structure capable of accommodating a part or all of the object 70. For example, when the object 70 is a human face, the mask structure 120 may be configured to accommodate the entire human face or the entire human head.

The mask structure 120 may be spaced apart from the object 70. In one example, the skin imaging apparatus 130 may be provided as built into the mask structure 120, or may be provided as an attachable/detachable type that may be attached to and detached from the mask structure 120. In one example, the skin imaging apparatus 130 may be or include the hyperspectral imaging system 100 illustrated in FIG. 1. The skin condition analyzing apparatus 200 may include a display device capable of displaying images captured by the skin imaging apparatus 130. This display device may be connected to a hyperspectral camera (e.g., 90 in FIG. 1) included in the skin imaging apparatus 130. The display device may be installed at a position where a user using the skin condition analyzing apparatus 200 may view a captured image of his or her skin in real time.

The disclosed imaging apparatus may include a line-illuminating light source and radiate linear light to an object. In addition, in the disclosed imaging apparatus, the light reflection unit provided at a position where the linear light emitted from the line-illuminating light source is first incident is rotatable about two axes perpendicular to each other, so as to the linear light may perform two-dimensional scanning of the object. Accordingly, the linear light radiated to the object may be irradiated according to a region of interest (analysis target area) of the object. In other words, an irradiation region of the linear light may be limited to a region of interest of the object. When the object is a human face or skin and the linear light is ultraviolet rays, the disclosed imaging apparatus may minimize the region of the face or skin exposed to ultraviolet rays.

Additionally, in the case of the disclosed imaging apparatus, a direction of the linear light radiated to the object and a direction of the camera for detecting the light incident from the object are coincide (parallel). Therefore, a side effect of reducing the intensity of light incident from the object to the camera, which may occur due to the discrepancy between the two directions, may be prevented.

Therefore, in the case of a skin analyzing apparatus using the disclosed imaging apparatus, a high-quality image of the region of interest of the skin may be obtained while minimizing ultraviolet rays radiated to the skin.

It should be understood that embodiments described herein should be considered in a descriptive sense only and not for purposes of limitation. Descriptions of features or aspects within each embodiment should typically be considered as available for other similar features or aspects in other embodiments. While one or more embodiments have been described with reference to the figures, it will be understood by those of ordinary skill in the art that various changes in form and details may be made therein without departing from the spirit and scope as defined by the following claims.

What is claimed is:

1. A line-illuminating light source comprising:
a light emitter;
a first lens provided at a position where light emitted from the light emitter is incident;
a slit comprising a linear opening through which the light incident from the first lens passes; and
a second lens provided at a position where the light passing through the linear opening is incident, and configured to direct the light toward an object.

2. The line-illuminating light source of claim 1, wherein the first lens is an aspherical lens.

3. The line-illuminating light source of claim 1, wherein the second lens is an achromatic lens.

4. The line-illuminating light source of claim 1, wherein the light emitter is configured to emit ultraviolet light.

5. The line-illuminating light source of claim 1, wherein the light emitter, the first lens, the slit, and the second lens are aligned on a same optical axis.

6. An imaging apparatus comprising:
a light source configured to emit linear light;
a light reflector configured to reflect the linear light incident from the light source;
a first filter configured to filter the linear light reflected from the light reflector;
a light transmission reflector configured to reflect the linear light passing through the first filter toward an object and transmit light incident from the object;
a second filter configured to filter the light that has passed through the light transmission reflector after being incident from the object; and
a camera configured to capture the light passing through the second filter, wherein the light transmission reflector, the second filter, and the camera are aligned on a same optical axis.

7. The imaging apparatus of claim 6, wherein the light source comprises:
a light emitter;
a first lens provided at a position where the light emitted from the light emitter is incident;
a slit comprising a linear opening through which the light incident from the first lens passes; and
a second lens provided at a position where the light passing through the linear opening is incident.

8. The imaging apparatus of claim 7, wherein the light emitter is configured to emit ultraviolet light.

9. The imaging apparatus of claim 6, wherein the camera comprises a hyperspectral image sensor.

10. The imaging apparatus of claim 6, wherein the light reflector is configured to rotate about two axes perpendicular to each other so that the linear light incident from the light source moves toward the object in two different perpendicular directions.

11. The imaging apparatus of claim 6, wherein the light transmission reflector is configured to reflect ultraviolet rays and transmit visible light.

12. The imaging apparatus of claim 6, wherein the light source, the light reflector, the first filter, and the light transmission reflector are aligned on a same optical axis.

13. The imaging apparatus of claim 6, wherein the light source, the light reflector, the first filter, the light transmission reflector, the second filter, and the camera are aligned so that light reflected from the light transmission reflector and radiated to the object and light incident from the object to the light transmission reflector due to the radiated light are parallel to each other.

14. The imaging apparatus of claim 6, wherein the light reflector comprises a folding mirror.

15. The imaging apparatus of claim 6, wherein the light reflector comprises a dichroic mirror.

16. The imaging apparatus of claim 6, wherein the first filter comprises an excitation filter.

17. The imaging apparatus of claim 6, wherein the second filter comprises an emission filter.

18. A skin analyzing apparatus comprising:
a mask structure configured to cover a surface of an object; and
the imaging apparatus of claim 6 provided in an region of the mask structure facing the surface of the object.

19. The skin analyzing apparatus of claim 18, wherein the imaging apparatus is mounted in the mask structure.

20. The skin analyzing apparatus of claim 18, wherein the imaging apparatus is attachable to and detachable from the mask structure.
